(19) United States
(12) Patent Application Publication
(10) Pub. No.: US 2016/0185882 A1
(43) Pub. Date: Jun. 30, 2016

(54) COMPOUND AND POLYMER PREPARED THEREFROM

(71) Applicant: INDUSTRIAL TECHNOLOGY RESEARCH INSTITUTE, Hsinchu (TW)

(72) Inventors: Yu-Lin CHU, New Taipei City (TW); Hsuan-Wei LEE, Pingtung City (TW); Chih-Hsiang LIN, Taipei City (TW); Chih-Feng HUANG, New Taipei City (TW); Yu-Min HAN, Taichung City (TW); Wen-Hua CHEN, Hsinchu City (TW)

(73) Assignee: INDUSTRIAL TECHNOLOGY RESEARCH INSTITUTE, Hsinchu (TW)

(21) Appl. No.: 14/849,907

(22) Filed: Sep. 10, 2015

(30) Foreign Application Priority Data
Dec. 25, 2014 (TW) 103145421

(51) Int. Cl. C08F 4/00; C08F 293/00; C07D 333/20; C08F 4/04

(52) U.S. Cl. CPC C08F 4/00; C08F 4/04; C08F 293/005; C07D 333/20

(57) ABSTRACT

A compound, and a polymer prepared therefrom, are provided. The compound has a structure represented by Formula (I) or Formula (II):

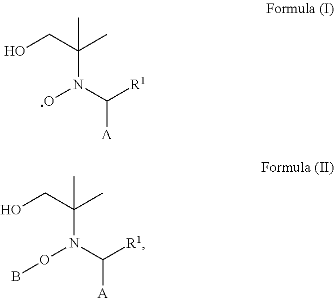

wherein A is

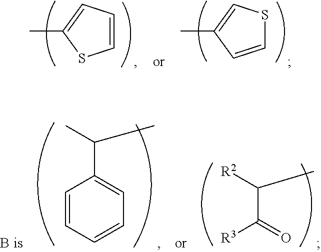

R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R² is H, or C₁₋₁₀ alkyl; and, R³ is C₁₋₁₀ alkoxy, C₁₋₁₀ alkanol, amine, or hydroxy.

### CROSS REFERENCE TO RELATED APPLICATION

The application is based on, and claims priority from, Taiwan Application Serial Number 103145421, filed on Dec. 25, 2014, the disclosure of which is hereby incorporated by reference herein in its entirety.

### TECHNICAL FIELD

The disclosure relates to a compound and a polymer prepared from the compound.

### BACKGROUND

Due to the superior optical and mechanical characteristics, vinyl-based polymers are widely used in the photovoltaic element, solar cell field, dispersants, coatings, and other functional additives.

At present, it is well known in the industry that vinyl-based polymers can be prepared by polymerizing the vinyl-based monomers via an active radical polymerization process. Specific examples for the active radical polymerization process include (i) nitroxide mediated polymerization (NMP); (ii) atom transfer radical polymerization (ATRP); (iii) reversible addition fragmentation chain transfer polymerization (RAFT); (iv) organotellurium mediated living radical polymerization (TERP); and, (v) reversible chain transfer catalyzed polymerization (RTCP). Among them, the nitroxide mediated polymerization (NMP) has been developed over a long period of time and come of age, thereby being widely applied in industrial applications. However, currently commercially available initiators of nitroxide mediated polymerization (NMP) have the disadvantages of high reaction temperature, low monomer conversion rate, and a narrow range of applicable monomers.

Therefore, a novel nitroxide initiator suitable for use in radical polymerization process is desired for solving the aforementioned problems.

### SUMMARY

According to embodiments of the disclosure, the disclosure provides a compound, which can be a stable radical compound. The compound has a structure represented by Formula (I), or Formula (II):

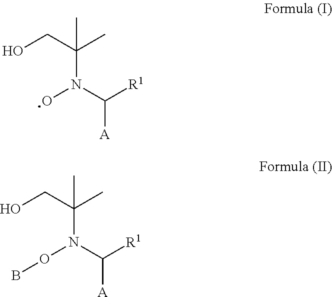

wherein, A is

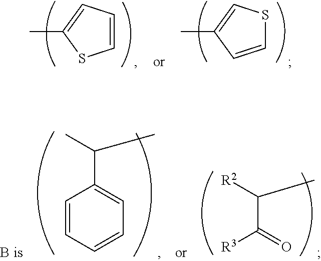

R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R² is H, or C₁₋₁₀ alkyl; and, R³ is C₁₋₁₀ alkoxy, C₁₋₁₀ alkanol, amine, or hydroxy.

According to another embodiment of the disclosure, the disclosure provides a polymer prepared from the aforementioned compound serving as an initiator. The polymer can be a reaction product of a composition, wherein the composition includes a first monomer, wherein the first monomer can be vinyl-based monomer and a compound having a structure represented by Formula (I), or Formula (II).

A detailed description is given in the following embodiments.

### DETAILED DESCRIPTION

This description is made for the purpose of illustrating the general principles of the disclosure and should not be taken in a limiting sense. The scope of the disclosure is determined by reference to the appended claims.

The disclosure provides a compound, and a polymer prepared from the compound. According to an embodiment of the disclosure, the compound can be a stable radical nitroxide. Since the compound of the disclosure has a polar group (i.e. thiophene), the C—O bond dissociation energy during a radical polymerization can be reduced when the compound of the disclosure serves as an initiator of the radical polymerization. Therefore, the radical polymerization employing the compound of the disclosure as an initiator can have a low reaction temperature (equal to or less than 90° C., such as between 90° C. and 25° C.), and the obtained polymer has a high monomer conversion rate (>50%). On the other hand, when the compound of the disclosure serves as an initiator of the nitroxide-mediated polymerization, the range of monomers, which can be used in the nitroxide-mediated polymerization, can be extended. For example, acrylate-based monomer and methacrylate-based monomer can serve as the monomer for performing a nitroxide-mediated polymerization (with a reaction temperature lower than 90° C.), when the compound of the disclosure serves as an initiator of the nitroxide-mediated polymerization.

According to an embodiment of the disclosure, the compound can have a structure represented by Formula (I) or Formula (II):

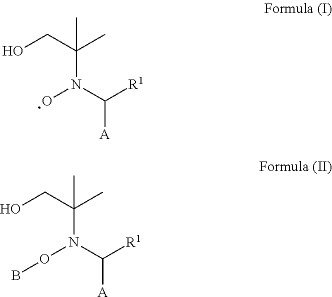

wherein, A is

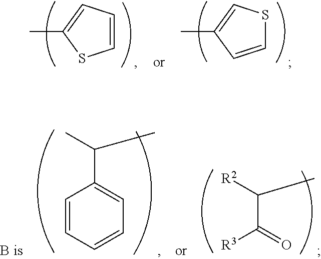

R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R² is H, or C₁₋₁₀ alkyl; and, R³ is C₁₋₁₀ alkoxy, C₁₋₁₀ alkanol, amine, or hydroxy.

According to an embodiment of the disclosure, the compound can have a structure represented by Formula (III) or Formula (IV):

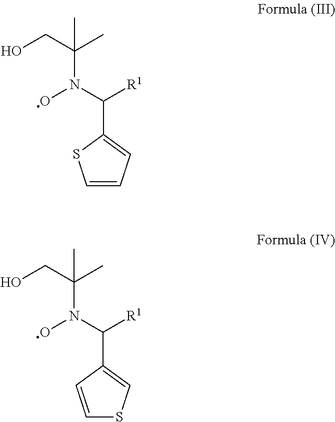

wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group. In particular, the oxygen atom marked with the symbol “•” means the oxygen atom has a radical, and the compound having a structure represented by Formula (III) or Formula (IV) is a stable radical compound.

According to an embodiment of the disclosure, R¹ can be methyl, ethyl, propyl, isopropyl, n-butyl, iso-butyl, tert-butyl, pentyl, hexyl, or cyclohexyl. In addition, according to other embodiments of the disclosure, R¹ can be phenyl, biphenyl, pyridyl, furyl, carbazole, naphthyl, anthryl, phenanthrenyl, imidazolyl, pyrimidinyl, quinolinyl, indolyl, or thiazolyl. According to some embodiments of the disclosure, R¹ can be methoxy, ethoxy, propoxy, isopropoxy, n-butoxy, iso-butoxy, tert-butoxy, pentoxy, or hexyloxy.

In addition, the compound having the structure represented by Formula (III) or Formula (IV) can further react with a styrene-based monomer, thus obtaining a unimolecular initiator compound having a structure represented by Formula (V), or Formula (VI):

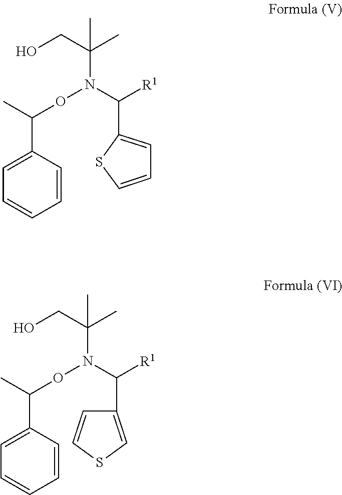

In addition, the compound having the structure represented by Formula (III) or Formula (IV) can further react with an acrylate-based monomer, or methacrylate-based monomer, thus obtaining an initiator having a structure represented by Formula (VII) or Formula (VIII):

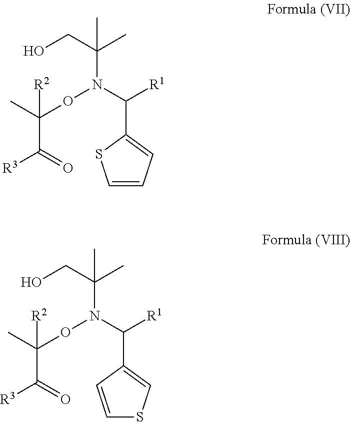

wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R² is H, or C₁₋₁₀ alkyl; and, R³ is C₁₋₁₀ alkoxy, C₁₋₁₀ alkanol, amine, or hydroxy.

According to an embodiment of the disclosure, the disclosure provides a polymer prepared from reacting the compound having a structure represented by Formula (I), or Formula (II) with an initiator and at least one monomer.

The polymer can be a reaction product of a composition, wherein the composition includes: (a) a first monomer, wherein the first monomer can be a vinyl-based monomer; (b) an initiator; and (c) a compound having a structure represented by Formula (III) or Formula (IV).

According to an embodiment of the disclosure, the molecular weight (such as number average molecular weight) of the polymer can be between about 4,800 and 500,000, such as between about 8,000 and 400,000, or between about 8,000 and 300,000. In addition, the polymer can have a polydispersity index (PDI) between about 1.1 and 1.5, wherein the polydispersity index of the polymer of the disclosure can be modified by adjusting the reaction temperature of the polymerization.

According to an embodiment of the disclosure, the composition for preparing the polymer of the disclosure can have 1-50,000 parts by mole (such as: 1-10,000 parts by mole, 1-6,000, or 1-3,000 parts by mole) of (a) the first monomer, 0.01-1 parts by mole of (b) the initiator, and 1 part by mole of (c) the compound having a structure represented by Formula (III) or Formula (IV). In addition, according to another embodiment of the disclosure, the composition can have (a) a first monomer, wherein the first monomer is a vinyl-based monomer; (b) optional addition of a compound having a structure represented by Formula (III) or (IV) for adjusting the concentration of radical; and (c) a compound having a structure represented by Formula (V), Formula (VI), Formula (VII), or Formula (VIII). According to an embodiment of the disclosure, the composition for preparing the polymer of the disclosure can have 1-50,000 parts by mole (such as 1-10,000 parts by mole, 1-6,000, or 1-3,000 parts by mole) of (a) the first monomer, 0-0.5 parts by mole (such as 0.01-0.5 parts by mole) of (b) the compound having a structure represented by Formula (III) or (IV), and 1 part by mole of (c) the compound having a structure represented by Formula (V), Formula (VI), Formula (VII), or Formula (VIII).

According to an embodiment of the disclosure, the first monomer can include acrylate-based monomer, methacrylate-based monomer, or styrene-based monomer. wherein the acrylate-based monomer can include methyl acrylate, ethyl acrylate, isopropyl, or butyl acrylate; the methacrylate-based monomer can include methyl methacrylate, ethyl methacrylate, propyl methacrylate, butyl methacrylate, benzyl methacrylate, hexyl methacrylate, cyclohexyl methacrylate, dodecyl methacrylate, or dimethylaminoethyl methacrylate; and, the styrene-based monomer can include styrene, α-methyl styrene, para-methyl styrene, meta-methyl styrene, ortho-methyl styrene, α-ethyl styrene, 2,4-dimethyl styrene, para-tert-butyl styrene, or α-methyl-para-methyl styrene.

According to an embodiment of the disclosure, wherein the initiator can include azo initiator, such as N,N′-azobisisobutyronitrile (AIBN), 2,2′-azobisisoheptonitrile (ABVN), 2,2′-azobis-2-methylbutyronitrile (AMBN), 1,1′-azobis(cyclohexane-1-carbonitrile) (ACCN), 1-[(cyano-1-methylethyl)azo] formamide (CABN) 2,2′-azobis (2-methylpropionamide)dihydrochloride (AIBA), dimethyl 2,2′-azobis(2-methylpropionate) (AIBME), or 2,2′-azobis[2-(2-imidazolin-2-yl)propane] dihydrochloride (AIBI). Furthermore, the initiator can include peroxide initiator (such as benzoyl peroxide (BPO), dicumyl peroxide (DCP), lauroyl peroxide (LPO), methyl ethyl ketone peroxide (MEKPO), t-butyl cumyl peroxide (tBCP), or a combination thereof.

According to an embodiment of the disclosure, the polymer of the disclosure can have a structure represented by Formula (IX), Formula (X), Formula (XI), or Formula (XII):

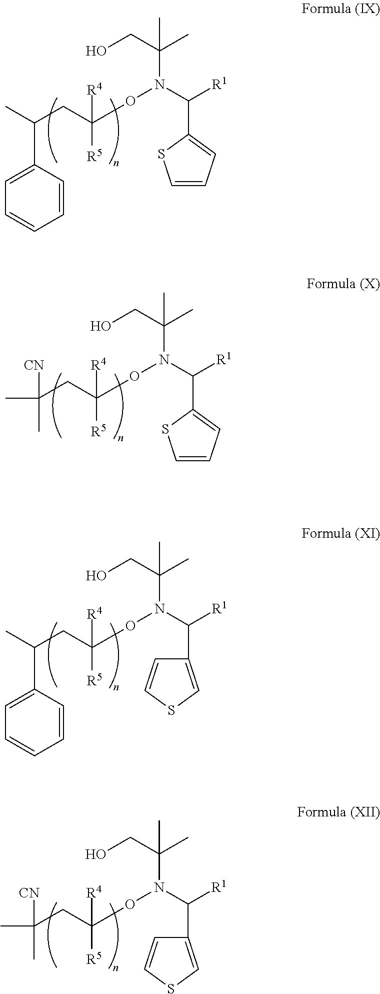

wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R⁴ is hydrogen, or C₁₋₆ alkyl; R⁵ is alkoxycarbonyl, or substituted or unsubstituted phenyl; and, n is larger than 1, such as between 1-100. For example, R⁴ can be methyl, ethyl, propyl, isopropyl, n-butyl, iso-butyl, tert-butyl, pentyl, or hexyl; and, R⁵ can be methoxycarbonyl, ethoxycarbonyl, propoxycarbonyl, butoxycarbonyl, benzyloxycarbonyl, dodecyloxy carbonyl, methylphenyl, dimethylphenyl, or butylphenyl.

According to an embodiment of the disclosure, the composition used for preparing the polymer of the disclosure can further include a second monomer, wherein the second monomer is different from the first monomer, and the second monomer can be a vinyl-based monomer.

In addition, according to other embodiments of the disclosure, the polymer having a structure represented by Formula (IX), Formula (X), Formula (XI), or Formula (XII) can be substituted for the compound having a structure represented by Formula (III), Formula (IV), Formula (V), Formula (VI), Formula (VII), or Formula (VIII) to react with a vinyl-based monomer, obtaining a block copolymer. Accordingly, the polymer of the disclosure can have a structure represented by Formula (XIII), Formula (XIV), Formula (XV), or Formula (XVI):

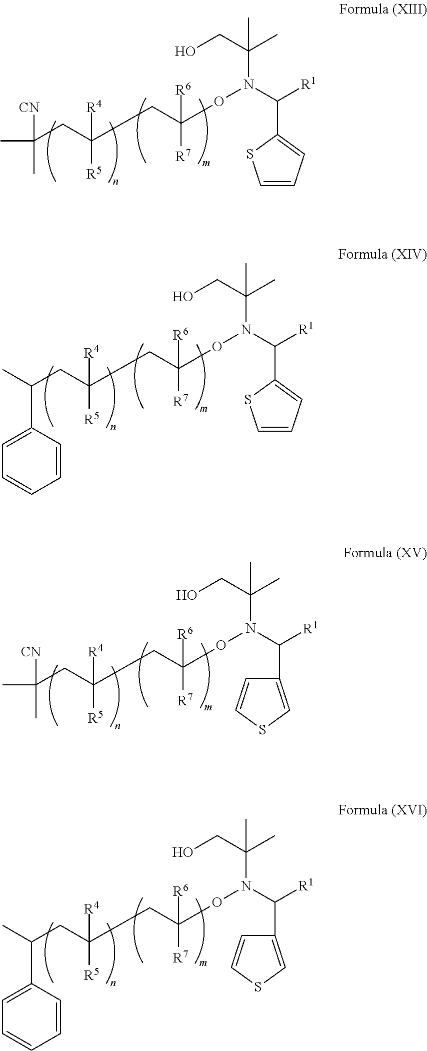

wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R⁴ is hydrogen, or C₁₋₆ alkyl; R⁵ is alkoxycarbonyl, or substituted or unsubstituted phenyl; R⁶ is hydrogen, or C₁₋₆ alkyl; R⁷ is alkoxycarbonyl, or substituted or unsubstituted phenyl, and R⁵ is different from the R⁷; and, n is larger than 1, such as between 1-100; and m is larger than 1, such as between 1-100. For example, R⁴ and R⁶ can be independently methyl, ethyl, propyl, isopropyl, n-butyl, iso-butyl, tert-butyl, pentyl, or hexyl; and, R⁵ and R⁷ can be independently methoxycarbonyl, ethoxycarbonyl, propoxycarbonyl, butoxycarbonyl, benzyloxycarbonyl, dodecyloxy carbonyl, methylphenyl, dimethylphenyl, or butylphenyl.

Below, exemplary embodiments will be described in detail so as to be easily realized by a person having ordinary knowledge in the art. The disclosure concept may be embodied in various forms without being limited to the exemplary embodiments set forth herein. Descriptions of well-known parts are omitted for clarity.

### Preparation of the Stable Radical Compound

### Example 1

First, methyl-2-nitropropan-1-ol (1 eq), pyridine (1 eq), and ethyl ether were added into a reaction bottle. After stirring, trimethylchlorosilane (TMSCl, 1 eq) was added into the reaction bottle at a temperature lower than 25° C. After reacting for 2 hours, the reaction bottle was warmed to 25° C. After filtration and purification by vacuum distillation, Compound (1), with a yield of 87.6%, was obtained. The synthesis pathway of the above reaction was as follows:

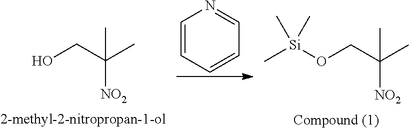

The measurement result of nuclear magnetic resonance spectrometry of Compound (1) is shown below: ¹H NMR (400 MHz, CDCl₃, 294 K): δ 3.83 (s, 2H), 1.55 (s, 6H), 0.01 (s, 9H).

Next, Compound (1) (1 eq), 2-methyl propanal (2 eq), and ammonium chloride (NH4Cl) (1.1 eq) were added into a reaction bottle, and water and ethyl ether serving as solvent were added into a reaction bottle. After stirring, zinc powder (4 eq) was added slowly into the reaction bottle at a temperature lower than 25° C. Next, after warming to 25, the mixture was stirred for 24 hours. After filtration, the filtrate was washed with methanol. After extraction with dichloromethane, concentration, and purification by column chromatography, Compound (2) was obtained, with a yield of 92%. The synthesis pathway of the above reaction was as follows:

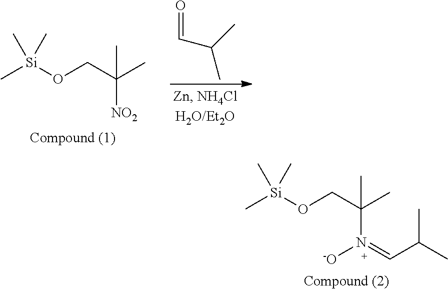

The measurement result of nuclear magnetic resonance spectrometry of Compound (2) is shown below: ¹H NMR (400 MHz, CDCl₃, 294 K): δ 6.31 (d, 1H), 3.61 (s, 2H), 3.10 (m, 1H), 1.36 (s, 6H), 1.02 (d, 6H), 0.01 (s, 9H).

Next, compound 2 (1 eq) and tetrahydrofuran (THF) were added into a reaction bottle. Next, thiophen-2-yl-magnesium bromide (2.8 eq) was slowly added into the reaction bottle, and the result was stirred at 25° C. for 24 hours. Next, ammonium chloride and water were added into the reaction bottle. The result was extracted three times by ethyl ether, and the organic phase was separated. Methanol, ammonia, and Cu(OAc)₂ (0.1 eq) were added into the reaction bottle, obtaining a brown solution. After introducing air into the reaction bottle until obtaining a dark-green solution, the result was extracted two times by chloroform. Next, after concentration, and purification by column chromatography, Compound (3), with a yield of 31%, was obtained. The synthesis pathway of the above reaction was as follows:

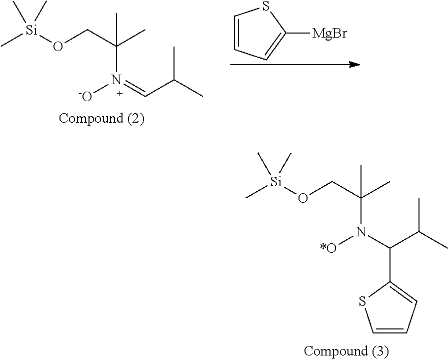

The measurement result of nuclear magnetic resonance spectrometry of Compound (3) is shown below: ¹H NMR (400 MHz, CDCl₃, 294 K): δ 7.5˜7.0 (m, 3H), 3.61 (s, 2H), 3.10 (m, 1H), 1.36 (s, 6H), 1.02 (d, 6H), 0.01 (s, 9H).

Next, Compound (3) (1 eq) and tetrabutylammonium fluoride (TBAF) (1.2 eq) were dissolved in tetrahydrofuran, and then added into a reaction bottle. Next, the reaction was stirred at 25° C. for 2 hours. Next, the result was extracted three times by water and dichloromethane. After concentration, Stable radical compound (1), with a yield of 72%, was obtained. The synthesis pathway of the above reaction was as follows:

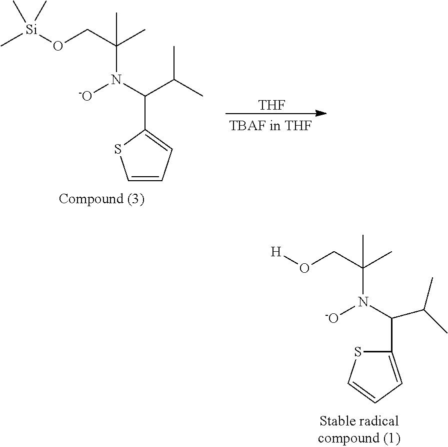

Stable radical compound (1) analyzed with infrared spectroscopy exhibits a characteristic peak of hydroxyl group (which is absent in the infrared spectrum of Compound (3)) at 3500 cm⁻¹. Furthermore, the characteristic peak (corresponding to Si—C bond) at 1250 cm⁻¹ is absent in the infrared spectrum of Stable radical compound (1).

### Example 2

Compound (2) (1 eq), and tetrahydrofuran (THF) were added into a reaction bottle. Next, thiophen-3-yl magnesium bromide (2.8 eq) was added into the reaction bottle, and stirred at 25° C. for 24 hours. Next, ammonium chloride and water were added into the reaction bottle. The result was extracted three times by ethyl ether, and the organic phase was separated. Methanol, ammonia, and Cu(OAc)₂ (0.1 eq) were added into the reaction bottle, obtaining a brown solution. After introducing air into the reaction bottle until obtaining a dark-green solution, the result was extracted two times by chloroform. Next, after concentration, and purification by column chromatography, Compound (4), with a yield of 31%, was obtained. The synthesis pathway of the above reaction was as follows:

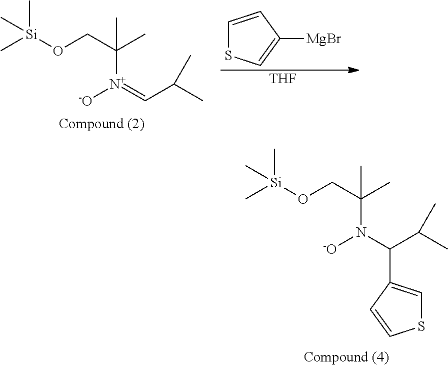

The measurement result of nuclear magnetic resonance spectrometry of Compound (4) is shown below: ¹H NMR (400 MHz, CDCl₃, 294 K): δ 7.5˜7.0 (m, 3H), 3.61 (s, 2H), 3.10 (m, 1H), 1.36 (s, 6H), 1.02 (d, 6H), 0.01 (s, 9H).

Next, Compound (4) (1 eq) and tetrabutylammonium fluoride (TBAF) (1.2 eq) were dissolved in tetrahydrofuran, and then added into a reaction bottle. Next, the reaction was stirred at 25° C. for 2 hours. Next, the result was extracted three times by water and dichloromethane. After concentration, Stable radical compound (2), with a yield of 72%, was obtained. The synthesis pathway of the above reaction was as follows:

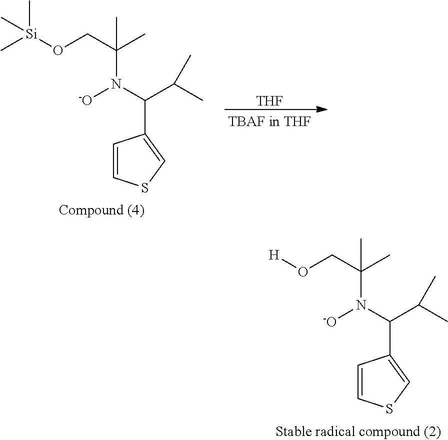

Stable radical compound (2) analyzed with infrared spectroscopy exhibits a characteristic peak of hydroxyl group (which is absent in the infrared spectrum of Compound (3)) at 3500 cm⁻¹. Furthermore, the characteristic peak (corresponding to Si—C bond) at 1250 cm⁻¹ is absent in the infrared spectrum of Stable radical compound (2).

### Preparation of Unimolecular Initiator Compound

### Example 3

Compound (4) (1 eq), styrene (2 eq), toluene, and ethanol were added into a reaction bottle. Next, Jacobsen's catalyst (0.2 eq), and sodium borohydride (3 eq) were sequentially added into the reaction bottle, and then the mixture was stirred for 24 hours within a bubble column reactor. After concentration, the result was extracted three times by dichloromethane and water. Next, after concentration, and purification by column chromatography, Compound (5), with a yield of 21%, was obtained. The synthesis pathway of the above reaction was as follows:

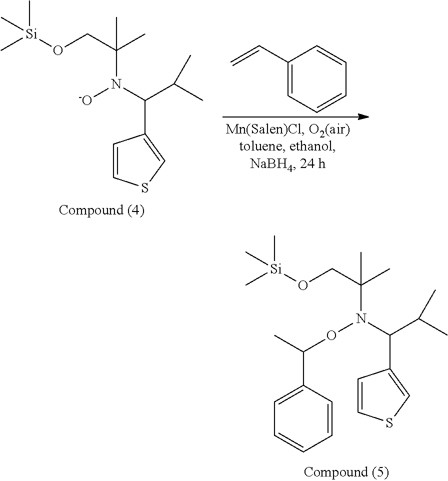

The measurement result of nuclear magnetic resonance spectrometry of Compound (5) is shown below: ¹H NMR (400 MHz, CDCl₃, 294 K): δ 7.43˜6.91 (m, 8H), 4.86 (t, 1H), 3.95˜3.72 (m, 3H), 2.96 (m, 1H), 1.68˜1.5 (m, 3H), 1.27 (s, 6H), 0.93 (d, 6H), 0.08 (s, 9H). Compound (5) analyzed with Fourier transform infrared spectroscopy exhibits characteristic peaks of thiophen group at 700, 841, and 1063 cm⁻¹, characteristic peaks corresponding to Si—C bond at 1092, and 1255 cm⁻¹, and characteristic peaks of aryl group at 1700-2000 cm⁻¹.

Next, Compound (5) (1 eq) and tetrabutylammonium fluoride (TBAF) (1.5 eq) were dissolved in tetrahydrofuran, and then added into a reaction bottle. Next, the reaction was stirred at 25° C. for 2 hours. Next, the result was extracted three times by water and dichloromethane. After concentration, Unimolecular initiator compound (1), with a yield of 75%, was obtained. The synthesis pathway of the above reaction was as follows:

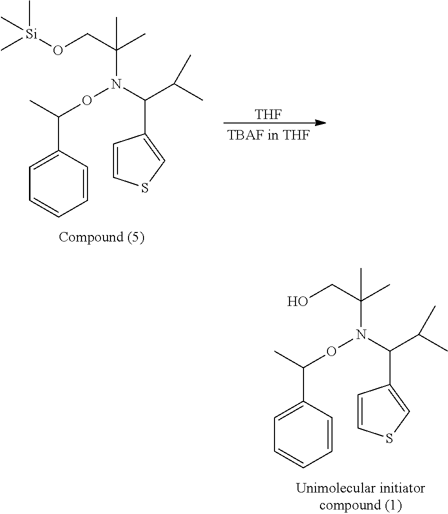 [SMILES: CC(ON(C(c1ccsc1)C(C)C)C(C)(C)CO[Si](C)(C)C)c1ccccc1 ; CC(ON(C(c1ccsc1)C(C)C)C(C)(C)CO)c1ccccc1 ; "THF"]

Unimolecular initiator compound (1) analyzed with infrared spectroscopy exhibits a characteristic peak of hydroxyl group (which is absent in the infrared spectrum of Compound (5)) at 3500 cm⁻¹. Furthermore, the characteristic peak (corresponding to Si—C bond) at 1255 cm⁻¹ is absent in the infrared spectrum of Unimolecular initiator compound (1).

### Example 4

Compound 3 (1 eq), styrene (2 eq), toluene, and ethanol were added into a reaction bottle. Next, Jacobsen's catalyst (0.2 eq), and sodium borohydride (3 eq) were sequentially added into the reaction bottle, and then the mixture was stirred for 24 hours within a bubble column reactor. After concentration, the result was extracted three times by dichloromethane and water. Next, after concentration, and purification by column chromatography, Compound (5), with a yield of 21%, was obtained. The synthesis pathway of the above reaction was as follows:

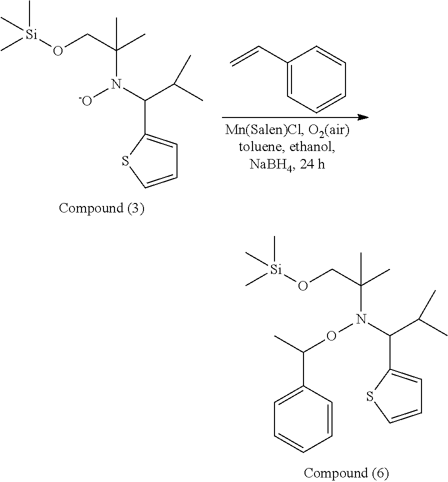

The measurement result of nuclear magnetic resonance spectrometry of Compound (6) is shown below: ¹H NMR (400 MHz, CDCl₃, 294 K): δ7.43˜6.91 (m, 8H), 4.86 (t, 1H), 3.95˜3.72 (m, 3H), 2.96 (m, 1H), 1.68˜1.5 (m, 3H), 1.27 (s, 6H), 0.93 (d, 6H), 0.08 (s, 9H). Compound (6) analyzed with Fourier transform infrared spectroscopy exhibits characteristic peaks of thiophen group at 700, 841, and 1063 cm⁻¹, characteristic peaks corresponding to Si—C bond at 1092, and 1255 cm⁻¹, and characteristic peaks of aryl group at 1700-2000 cm⁻¹.

Next, Compound 6 (1 eq) and tetrabutylammonium fluoride (TBAF) (1.5 eq) were dissolved in tetrahydrofuran, and then added into a reaction bottle. Next, the reaction was stirred at 25° C. for 2 hours. Next, the result was extracted three times by water and dichloromethane. After concentration, Unimolecular initiator compound (2), with a yield of 75%, was obtained.

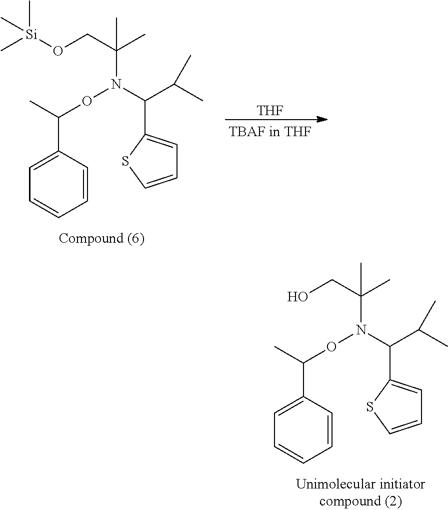 [SMILES: CC(ON(C(c1cccs1)C(C)C)C(C)(C)CO[Si](C)(C)C)c1ccccc1 ; CC(ON(C(c1cccs1)C(C)C)C(C)(C)CO)c1ccccc1 ; "THF"]

Unimolecular initiator compound (2) analyzed with infrared spectroscopy exhibits a characteristic peak of hydroxyl group (which is absent in the infrared spectrum of Compound (6)) at 3500 cm⁻¹. Furthermore, the characteristic peak (corresponding to Si—C bond) at 1255 cm⁻¹ is absent in the infrared spectrum of Unimolecular initiator compound (2).

### Preparation of Polymer

### Example 5

Stable radical compound (1) (1 eq), 2,2′-Azobisisobutyronitrile (AIBN) (0.5 eq), and styrene (100 eq) were added into a reaction bottle. After introducing nitrogen gas for exhausting oxygen, the mixture was stirred at 80° C. for 12 hours. After precipitation with methanol, Polymer (1) was obtained. The synthesis pathway of the above reaction was as follows:

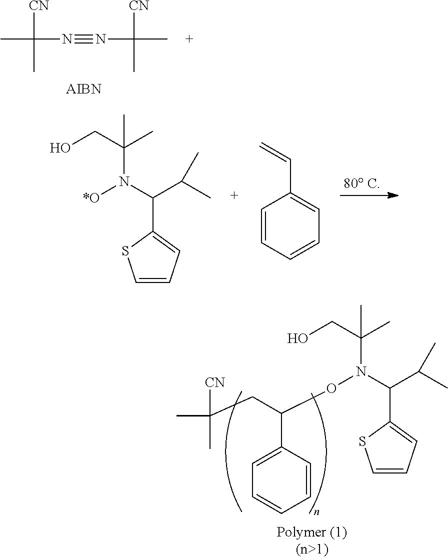

Next, the characteristics of Polymer (1) were measured by a gel permeation chromatography (GPC), and the results are shown in Table 1.

### Example 6

Polymer (1) (1.0 g, 1.0 eq), and styrene (1.3 g, 300 eq) were added into a reaction bottle. After introducing nitrogen gas for exhausting oxygen, the mixture was stirred at 80° C. for 12 hours. After precipitation with methanol, Polymer (2) was obtained. The synthesis pathway of the above reaction was as follows:

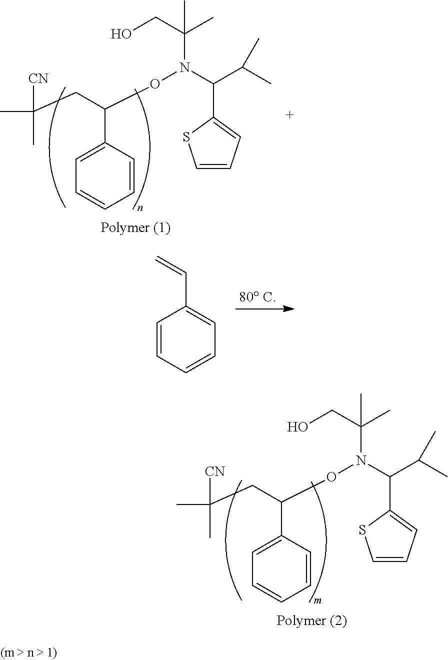

Next, the characteristics of Polymer (2) were measured by a gel permeation chromatography (GPC), and the results are shown in Table 1.

### Example 7

Unimolecular initiator compound (1) (1 eq), and styrene (100 eq) were added into a reaction bottle. After introducing nitrogen gas for exhausting oxygen, the mixture was stirred at 70° C. for 24 hours. After precipitation with methanol, Polymer (3) was obtained. The synthesis pathway of the above reaction was as follows:

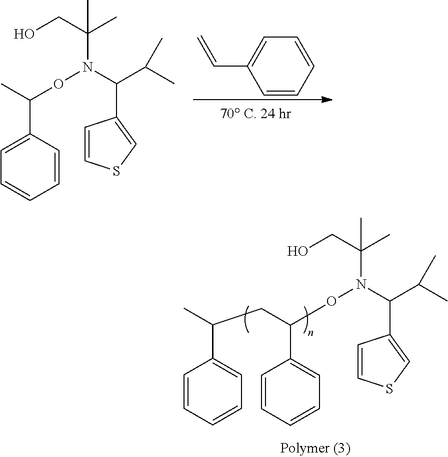

Next, the characteristics of Polymer (3) were measured by a gel permeation chromatography (GPC), and the results are shown in Table 1.

### Comparative Example 1

TIPNO (having a structure represented by

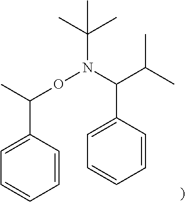 [SMILES: CC(ON(C(c1ccccc1)C(C)C)C(C)(C)C)c1ccccc1]

(1 eq), and styrene (100 eq) were added into a reaction bottle. After introducing nitrogen gas for exhausting oxygen, the mixture was stirred at 80° C. for 16 hours. After precipitation with methanol, Polymer (4) was obtained. The synthesis pathway of the above reaction was as follows:

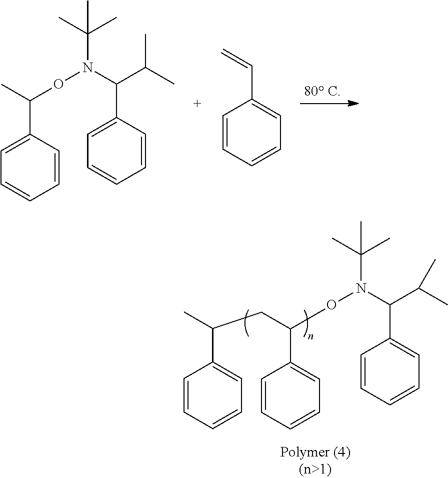

Next, the characteristics of Polymer (4) were measured by a gel permeation chromatography (GPC), and the results are shown in Table 1.

TABLE 1

| | | | | | weight | number | |
|---|---|---|---|---|---|---|---|
| | | | reaction | reaction | average | average | |
| | | | temperature | time | molecular | molecular | |
| | initiator | monomer | (° C.) | (hour) | weight (Mw) | weight(Mn) | PDI |
| Example 5 | Stable radical | styrene | 80 | 12 | 37797 | 25182 | 1.50 |
| | compound | | | | | | |
| | (1)/AIBN | | | | | | |
| Example 6 | Polymer (1) | styrene | 80 | 12 | 51279 | 41042 | 1.25 |
| Example 7 | Unimolecular | styrene | 70 | 24 | 6419 | 4943 | 1.29 |
| | initiator | | | | | | |
| | compound (1) | | | | | | |
| Comparative | TIPNO | styrene | 80 | 16 | 1205 | 910 | 1.32 |
| Example 1 | | | | | | | |

As shown in Table 1, the radical polymerizations disclosed in Example 5 and Example 6 can be performed at a relatively low reaction temperature (80° C.), and have a high monomer conversion rate (about 60%, determined by a gas chromatograph/mass spectrometer (GC-MS)). Furthermore, Polymers (1) and (2) have number average molecular weights (Mn) larger than 25000. The radical polymerizations disclosed in Example 7 can be performed at a relatively low reaction temperature (70° C.), and have a monomer conversion rate of about 25%. Furthermore, Polymer (3) has a number average molecular weight (Mn) of 4943. On the other hand, although the radical polymerizations disclosed in Comparative Example 1 can be performed at 80° C., they have a monomer conversion rate of less than 5%. Therefore, Polymer (4) has a number average molecular weight (Mn) of less than 1000. Accordingly, radical polymerizations employing the stable radical compound of the disclosure/AIBN or Unimolecular initiator compound of the disclosure can be performed at low temperatures and have a high monomer conversion rate. In addition, as shown in Example 6, Polymer (1) prepared by Example 5 can serve as an initiator for polymerizing styrene via a radical polymerization.

### Example 8

Stable radical compound (1) (2 eq), 2,2′-Azobisisobutyronitrile (AIBN) (1 eq), and n-butyl acrylate (200 eq) were added into a reaction bottle. After introducing nitrogen gas for exhausting oxygen, the mixture was stirred at 75° C. for 2 hours. After precipitation with methanol, Polymer (5) was obtained. The synthesis pathway of the above reaction was as follows:

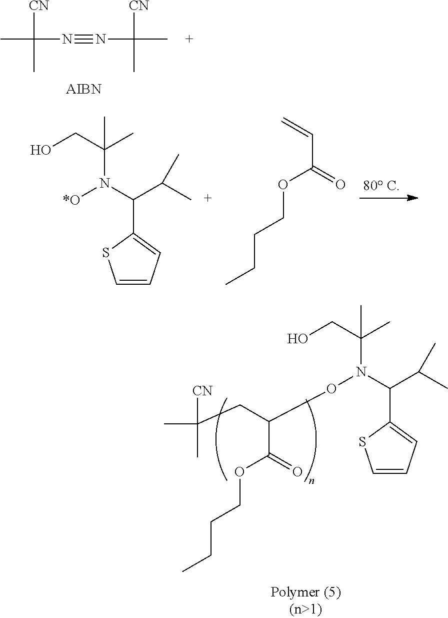

Next, the characteristics of Polymer (5) were measured by a gel permeation chromatography (GPC), and the results are shown in Table 2.

### Comparative Example 2

TIPNO (1 eq) (having a structure represented by

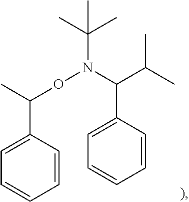 [SMILES: CC(ON(C(c1ccccc1)C(C)C)C(C)(C)C)c1ccccc1]

TIPNO radical (having a structure represented by

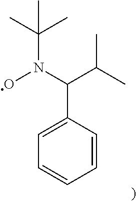

(0.05 eq), and n-butyl acrylate (200 eq) were added into a reaction bottle. After introducing nitrogen gas for exhausting oxygen, the mixture was stirred at 75° C. for 16 hours. No polymerization occurred.

### Comparative Example 3

TIPNO (1 eq) (having a structure represented by

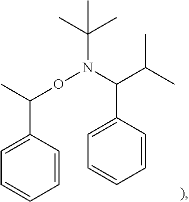 [SMILES: CC(ON(C(c1ccccc1)C(C)C)C(C)(C)C)c1ccccc1]

TIPNO radical (0.05 eq), and n-butyl acrylate (200 eq) were added into a reaction bottle. After introducing nitrogen gas for exhausting oxygen, the mixture was stirred at 125° C. for 24 hours. After precipitation with methanol, Polymer (6) was obtained. The synthesis pathway of the above reaction was as follows:

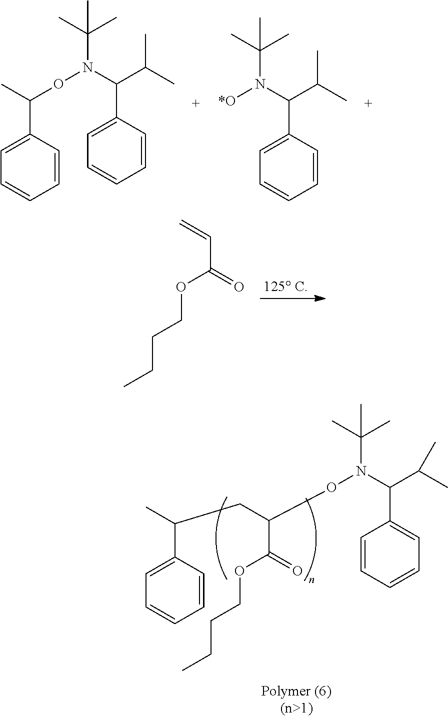

Next, the characteristics of Polymer (6) were measured by a gel permeation chromatography (GPC), and the results are shown in Table 2.

TABLE 2

| | | | | | weight | number | |
|---|---|---|---|---|---|---|---|
| | | | reaction | reaction | average | average | |
| | | | temperature | time | molecular | molecular | |
| | initiator | monomer | (° C.) | (hour) | weight (Mw) | weight(Mn) | PDI |
| Example 8 | Stable radical | n-butyl | 75 | 2 | 59412 | 48797 | 1.21 |
| | compound | acrylate | | | | | |
| | (1)/AIBN | | | | | | |
| Comparative | TIPNO | n-butyl | 75 | 16 | No reaction | | |
| Example 2 | | acrylate | | | | | |
| Comparative | TIPNO | n-butyl | 125 | 24 | 18371 | 14981 | 1.23 |
| Example 3 | | acrylate | | | | | |

As shown in Table 2, n-butyl acrylate monomer cannot be polymerized at 75° C. with TIPNO as initiator (Comparative Example 2) due to the high C—O bond dissociation energy of TIPNO, and the mixture of n-butyl acrylate, TIPNO, and TIPNO radical underwent a radical polymerization at 125° C. with a monomer conversion rate of about 58% (Comparative Example 3). On the other hand, the radical polymerization disclosed in Example 8 can be performed at a relatively low reaction temperature (75° C.), and have a high monomer conversion rate of about 61%, since the intermediate formed from the stable radical compound of the disclosure, AIBN, and n-butyl acrylate has a low C—O bond dissociation energy. Therefore, radical polymerizations employing the stable radical compound of the disclosure can be performed at low temperatures with high monomer conversion rate.

### Example 9

Stable radical compound (1) (2 eq), 2,2′-Azobisisobutyronitrile (AIBN)(0.5 eq), and methyl methacrylate (MMA) (100 eq) were added into a reaction bottle. After introducing nitrogen gas for exhausting oxygen, the mixture was stirred at 75° C. for 2.5 hours. After precipitation with methanol, polymer (7) was obtained. The synthesis pathway of the above reaction was as follows:

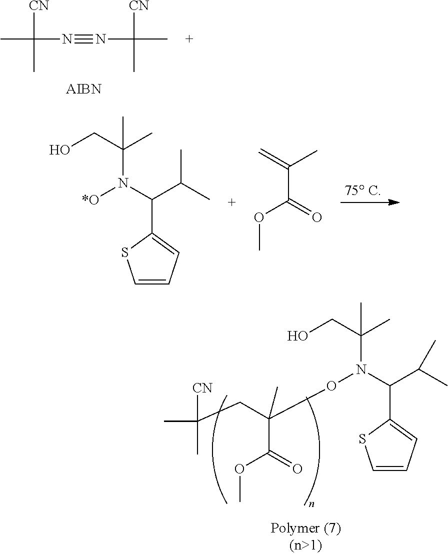

Next, the characteristics of Polymer (7) were measured by a gel permeation chromatography (GPC), and the results are shown in Table 3.

### Example 10

Polymer (7) (0.6 g, 1 eq), and styrene (5 g, 2800 eq) were added into a reaction bottle. After introducing nitrogen gas for exhausting oxygen, the mixture was stirred at 85° C. for 16 hours. After precipitation with methanol, polymer (8). The synthesis pathway of the above reaction was as follows:

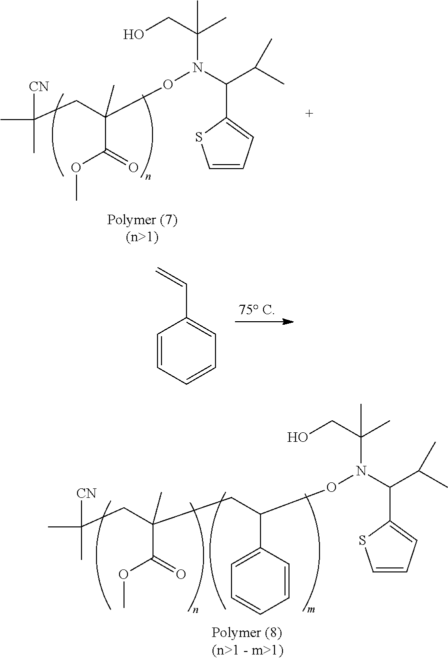

Next, the characteristics of Polymer (8) were measured by a gel permeation chromatography (GPC), and the results are shown in Table 3.

### Comparative Example 4

TIPNO (1 eq) (having a structure represented by

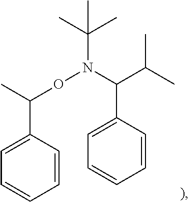 [SMILES: CC(ON(C(c1ccccc1)C(C)C)C(C)(C)C)c1ccccc1]

TIPNO radical (0.05 eq), and methyl methacrylate (MMA) (100 eq) were added into a reaction bottle. After introducing nitrogen gas for exhausting oxygen, the mixture was stirred at 75° C. for 12 hours. No polymerization occurred.

### Comparative Example 5

TIPNO (1 eq) (having a structure represented by

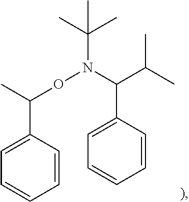 [SMILES: CC(ON(C(c1ccccc1)C(C)C)C(C)(C)C)c1ccccc1]

TIPNO radical (0.05 eq), and methyl methacrylate (MMA) (100 eq) were added into a reaction bottle. After introducing nitrogen gas for exhausting oxygen, the mixture was stirred at 125° C. for 24 hours. After precipitation with methanol, polymer (9) was obtained. The synthesis pathway of the above reaction was as follows:

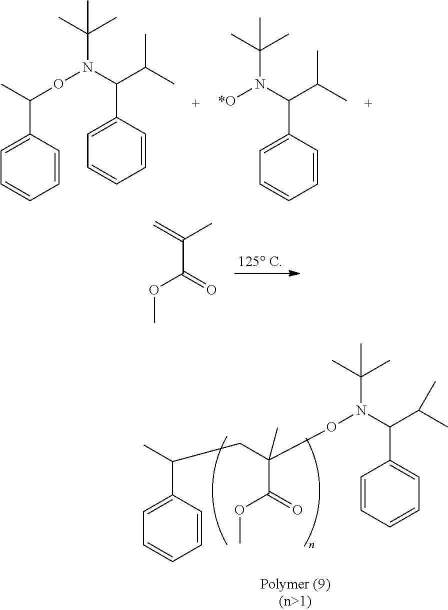

Next, the characteristics of Polymer (9) were measured by a gel permeation chromatography (GPC), and the results are shown in Table 3.

TABLE 3

| | | | | | weight | number | |
|---|---|---|---|---|---|---|---|
| | | | reaction | reaction | average | average | |
| | | | temperature | time | molecular | molecular | |
| | initiator | monomer | (° C.) | (hour) | weight (Mw) | weight(Mn) | PDI |
| Example 9 | Stable radical | methyl | 75 | 2.5 | 46109 | 35873 | 1.28 |
| | compound | methacrylate | | | | | |
| | (1)/AIBN | | | | | | |
| Example 10 | Polymer | styrene | 85 | 16 | 65906 | 54291 | 1.22 |
| | (7) | | | | | | |
| Comparative | TIPNO | methyl | 75 | 12 | No reaction | | |
| Example 4 | | methacrylate | | | | | |
| Comparative | TIPNO | methyl | 125 | 24 | 9218 | 5822 | 1.58 |
| Example 5 | | methacrylate | | | | | |

As shown in Table 3, methyl methacrylate monomer cannot be polymerized at 75° C. with TIPNO as initiator (Comparative Example 4) due to the high C—O bond dissociation energy of TIPNO, and the mixture of methyl methacrylate, TIPNO, and TIPNO radical underwent a radical polymerization at 125° C. with a monomer conversion rate of about 58% and a PDI of 1.58 (Comparative Example 5). On the other hand, the radical polymerization disclosed in Example 9 can be performed at a relatively low reaction temperature (75), and have a monomer conversion rate of about 48%, since the intermediate formed from the stable radical compound of the disclosure, AIBN, and methyl methacrylate has a low C—O bond dissociation energy. Therefore, the radical polymerization of Example 9 can be performed at low temperatures with an acceptable monomer conversion rate and a relatively narrow PDI (1.28). In addition, as shown in Example 10, Polymer (7) prepared by Example 9 can serve as an initiator for polymerizing styrene to obtain a block copolymer via a radical polymerization.

Accordingly, due to the polar thiophene group, the compound of the disclosure, having low C—O bond dissociation energy, can serve as an initiator used in the radical polymerization with low reaction temperature (less than 90° C.). Furthermore, the radical polymerization employing the compound of the disclosure can have a high monomer conversion rate (larger than 50%). On the other hand, when the compound of the disclosure serves as an initiator of the nitroxide-mediated polymerization, the range of monomers used in the nitroxide-mediated polymerization can be extended. For example, monomers, such as acrylate-based monomer or methacrylate-based monomer, can be polymerized at a reaction temperature lower than 90° C. in the presence of the compound of the disclosure.

It will be clear that various modifications and variations can be made to the disclosed methods and materials. It is intended that the specification and examples be considered as exemplary only, with a true scope of the disclosure being indicated by the following claims and their equivalents.

1. A compound, having a structure represented by Formula (I), or Formula (II): 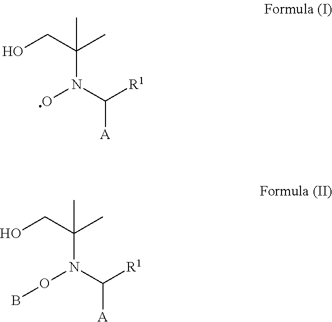
wherein, A is 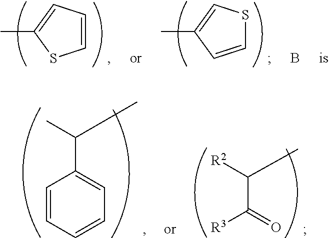
R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R² is H, or C₁₋₁₀ alkyl; and, R³ is C₁₋₁₀ alkoxy, C₁₋₁₀ alkanol, amine, or hydroxy.

2. The compound as claimed in claim 1, wherein the compound has the structure represented by Formula (III), or Formula (IV): 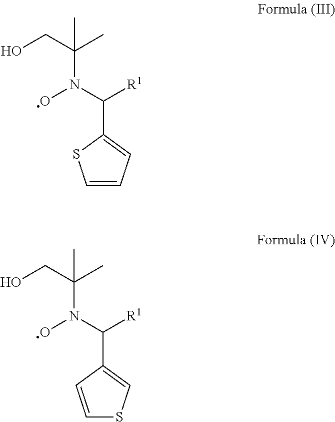
wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group.

3. The compound as claimed in claim 1, wherein the compound has the structure represented by Formula (V), Formula (VI), Formula (VII), or Formula (VIII): 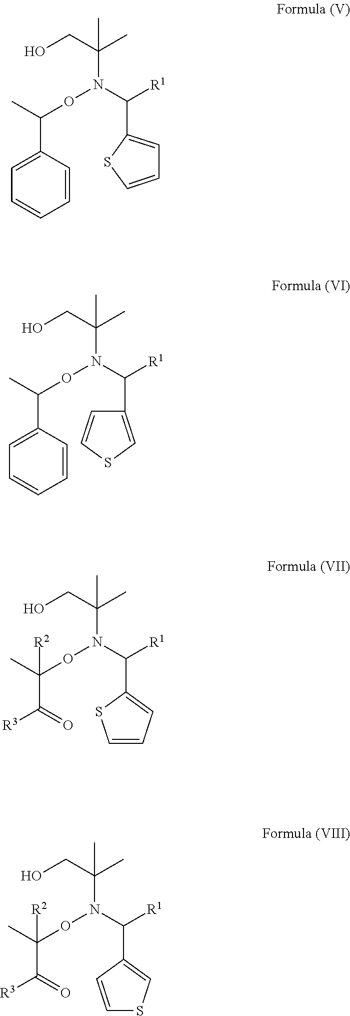
wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R² is H, or C₁₋₁₀ alkyl; and, R³ is C₁₋₁₀ alkoxy, C₁₋₁₀ alkanol, amine, or hydroxy.

4. The compound as claimed in claim 1, wherein R¹ is methyl, ethyl, propyl, isopropyl, n-butyl, iso-butyl, tert-butyl, pentyl, hexyl, or cyclohexyl.

5. The compound as claimed in claim 1, wherein R¹ is phenyl, biphenyl, pyridyl, furyl, carbazole, naphthyl, anthryl, phenanthrenyl, imidazolyl, pyrimidinyl, quinolinyl, indolyl, or thiazolyl.

6. The compound as claimed in claim 1, wherein R¹ is methoxy, ethoxy, propoxy, isopropoxy, n-butoxy, iso-butoxy, tert-butoxy, pentoxy, or hexyloxy.

7. A polymer, which is a reaction product of a composition, wherein the composition comprises:
a first monomer, wherein the first monomer is a vinyl-based monomer; and
the compound as claimed in claim 1.

8. The polymer as claimed in claim 7, wherein the composition further comprises an initiator, when the compound has a structure represented by Formula (I), or Formula (II).

9. The polymer as claimed in claim 7, wherein the first monomer comprises acrylate-based monomer, methacrylate-based monomer, or styrene-based monomer.

10. The polymer as claimed in claim 9, wherein the acrylate-based monomer comprises methyl acrylate, ethyl acrylate, isopropyl, or butyl acrylate.

11. The polymer as claimed in claim 9, wherein the methacrylate-based monomer comprises methyl methacrylate, ethyl methacrylate, propyl methacrylate, butyl methacrylate, benzyl methacrylate, hexyl methacrylate, cyclohexyl methacrylate, dodecyl methacrylate, or dimethylaminoethyl methacrylate.

12. The polymer as claimed in claim 9, wherein the styrene-based monomer comprises styrene, α-methyl styrene, para-methyl styrene, meta-methyl styrene, ortho-methyl styrene, α-ethyl styrene, 2,4-dimethyl styrene, para-tert-butyl styrene, or α-methyl-para-methyl styrene.

13. The polymer as claimed in claim 8, wherein the initiator comprises N,N′-azobisisobutyronitrile (AIBN), 2,2′-azobisisoheptonitrile (ABVN), 2,2′-azobis-2-methylbutyronitrile (AMBN), 1,1′-azobis(cyclohexane-1-carbonitrile) (ACCN), 1-[(cyano-1-methylethyl)azo]formamide (CABN), 2,2′-azobis(2-methylpropionamide)dihydrochloride (AIBA), dimethyl 2,2′-azobis(2-methylpropionate) (AIBME), 2,2′-azobis[2-(2-imidazol in-2-yl)propane] dihydrochloride (AIBI), benzoyl peroxide (BPO), dicumyl peroxide (DCP), lauroyl peroxide (LPO), methyl ethyl ketone peroxide (MEKPO), t-butyl cumyl peroxide (tBCP), or a combination thereof.

14. The polymer as claimed in claim 7, wherein the composition further comprises:
a second monomer, wherein the second monomer is different from the first monomer, and the second monomer is a vinyl-based monomer.

15. The polymer as claimed in claim 14, wherein the second monomer comprises acrylate-based monomer, methacrylate-based monomer, or styrene-based monomer.

16. The polymer as claimed in claim 15, wherein the acrylate-based monomer comprises methyl acrylate, ethyl acrylate, isopropyl, or butyl acrylate.

17. The polymer as claimed in claim 15, wherein the methacrylate-based monomer comprises methyl methacrylate, ethyl methacrylate, propyl methacrylate, butyl methacrylate, benzyl methacrylate, hexyl methacrylate, cyclohexyl methacrylate, dodecyl methacrylate, or dimethylaminoethyl methacrylate.

18. The polymer as claimed in claim 15, wherein the styrene-based monomer comprises styrene, α-methyl styrene, para-methyl styrene, meta-methyl styrene, ortho-methyl styrene, α-ethyl styrene, 2,4-dimethyl styrene, para-tert-butyl styrene, or α-methyl-para-methyl styrene.

19. The polymer as claimed in claim 7, wherein the polymer has a structure represented by Formula (IX), Formula (X), Formula (XI), or Formula (XII): 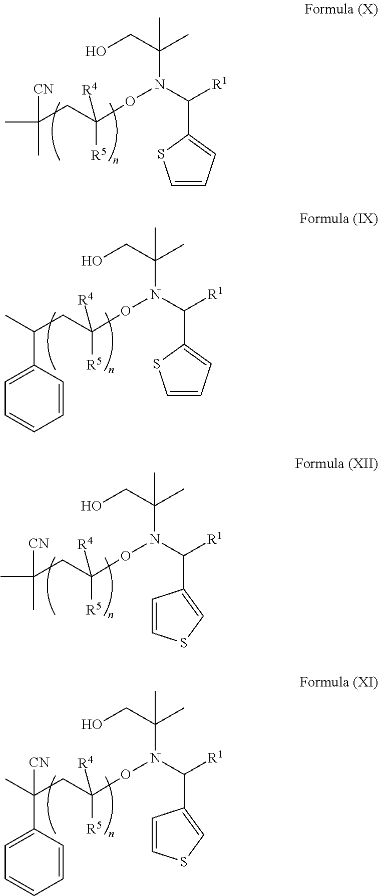
wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group; R⁴ is hydrogen, or C₁₋₆ alkyl; R⁵ is alkoxycarbonyl, or substituted or unsubstituted phenyl; and, n is larger than 1.

20. The polymer as claimed in claim 19, wherein R⁴ is methyl, ethyl, propyl, isopropyl, n-butyl, iso-butyl, tert-butyl, pentyl, or hexyl.

21. The polymer as claimed in claim 19, wherein R⁵ is methoxycarbonyl, ethoxycarbonyl, propoxycarbonyl, butoxycarbonyl, benzyloxycarbonyl, dodecyloxy carbonyl, methylphenyl, dimethylphenyl, or butylphenyl.

22. The polymer as claimed in claim 7, wherein the polymer has a structure represented by Formula (XIII), Formula (XIV), Formula (XV), or Formula (XVI): 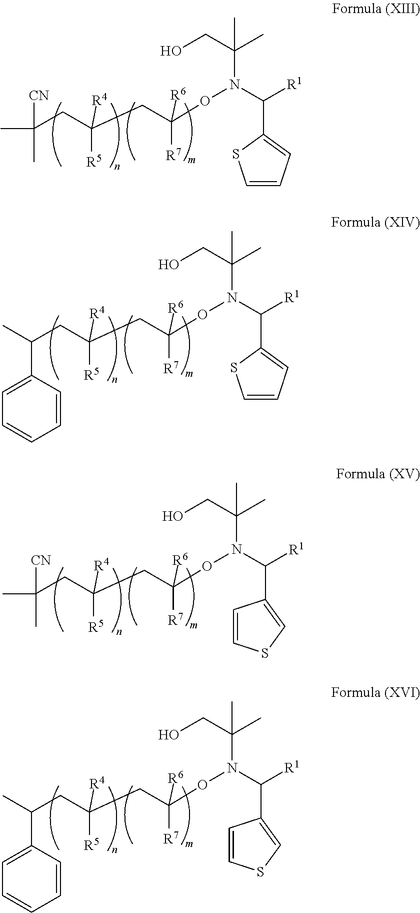
wherein, R¹ is C₁₋₁₀ alkyl, C₅₋₁₂ cycloalkyl, C₆₋₁₄ aryl, C₃₋₁₂ heteroaryl, C₁₋₁₀ alkoxy, C₆₋₁₂ aryloxy, C₁₋₁₀ silyl, amino, thiol, or phosphonate group;
R⁴ is hydrogen, or C₁₋₆ alkyl; R⁵ is alkoxycarbonyl, or substituted or unsubstituted phenyl; R⁶ is hydrogen, or C₁₋₆ alkyl; R⁷ is alkoxycarbonyl, or substituted or unsubstituted phenyl, and R⁵ is different from the R⁷; and, n is larger than 1, and m is larger than 1.

23. The polymer as claimed in claim 22, wherein R⁶ is methyl, ethyl, propyl, isopropyl, n-butyl, iso-butyl, tert-butyl, pentyl, or hexyl.

24. The polymer as claimed in claim 22, wherein R⁷ is methoxycarbonyl, ethoxycarbonyl, propoxycarbonyl, butoxycarbonyl, benzyloxycarbonyl, dodecyloxy carbonyl, methylphenyl, dimethylphenyl, or butylphenyl.
